(19) United States
(12) Patent Application Publication
(10) Pub. No.: US 2005/0084519 A1
(43) Pub. Date: Apr. 21, 2005

(54) Feed for fish and shellfish, additive therefor, and method for producing additive

(75) Inventor: Teruo Miyazaki, Suzuka-shi (JP)

Correspondence Address: JORDAN AND HAMBURG LLP, 122 EAST 42ND STREET, SUITE 4000, NEW YORK, NY, 10168, US

(73) Assignee: JAPAN PET DRUGS CO., LTD., Katsushika-ku (JP)

(21) Appl. No.: 10/734,011

(22) Filed: Dec. 11, 2003

(30) Foreign Application Priority Data
Oct. 17, 2003 (JP) 2003-357395

(51) Int. Cl. A61K 39/295; A23K 1/165; A61K 39/02; A61K 39/12

(52) U.S. Cl. 424/442; 424/200.1; 424/201.1; 424/199.1

(57) ABSTRACT

The additive of the present invention comprises a multitude of oil globules embedding biologically activating substances of the living fish and shellfish. The oil globules can be effectively absorbed through bowels of fish and shellfish. The additive may be produced by the method comprising mixing water with biologically activating substance; adding edible oil and a lipophilic emulsifier to the mixture of the water and the substance to produce a mixed solution; turning the oil phase of the mixed solution into oil globules embedding the substance by a homo-mixer or ultrasonic vibrations to produce an oil-in-water emulsion; and adding a hydrophilic emulsifier to the emulsion for stabilization.

### FIELD OF THE INVENTION

The present invention relates to feed for fish and shellfish, an additive for the feed, and a method for producing the feed and additive. The fish and shellfish throughout the present specification and claims include various aquatic animals such as fishes, shells, crabs, and shrimps/prawns. The feed includes feed for cultivation and feed for fish for viewing. The additive, as one of the subject matters of the present invention is a material or substance having an action of activating biological functions of fish and shellfish as well as immunopotentiating power.

### BACKGROUND OF THE INVENTION

In recent years, cultivation of fish and shellfish has been carried out worldwide in a large scale as natural living aquatic resources have decreased. Breeding fish for viewing has also been popular. It is conceived that keeping fish in an aquarium is categorized as one way of breeding fish for viewing.

Fish under the control of breeders for cultivation and breeding for the purpose of viewing are vulnerable to diseases. Presumably, a primary reason for this tendency is because fish and shellfish are bred in an overpopulated state in preserves, fishponds, or water tanks. Once a fish is infected with a disease, instantaneously, the other fish and shellfish in the same environment are infected with the disease. In most cases, the fish in the preserve or the fishpond may completely die out.

A further study on the causes of a high infection rate with pathogenic bacteria or virus in cultured fish revealed that the vitality of cultured fish and shellfish is low compared with naturally grown fish and shellfish and accordingly, the immunity of the former to pathogenic bacteria or virus is low compared with that of the latter. The causes for such low vitality of cultured fish and shellfish presumably include lack of exercise and highly stressful environment, deterioration of water quality resulting from the overpopulated cultivation, or the like.

Providing a measure against infection of a disease among cultured fish and shellfish is an urgent matter for breeders, and therefore, preventive measures have been studied. Japanese Unexamined Patent Publication No. 2001-86895 discloses a method for making a scar on the surface of fish with an instrument such as a needle for a syringe, and applying a medicament into the fish body through the scar, and an instrument for use in the application of the medicament.

The above technique, however, requires enormous labor and cost in carrying out the inoculation. Further, it is difficult to apply the technique to young fish. In addition, this technique is stressful to fish. In view of these various drawbacks, the proposed method does not provide versatile applicability.

It has been a customary practice to add a medicament and/or a dietary supplement to feed. This idea does not present a serious problem because medicaments and dietary supplements are supplied by way of natural feeding. However, merely mixing a medicament or a dietary supplement with feed is not effective because the medicament and the dietary supplement are decomposed by gastric juice secreted from fish or may likely be discharged without being absorbed. Thus, this method is not advantageous in the aspect of absorption efficiency.

In view of the above, the present invention has been made to improve the current situation relating to feed. An object of the present invention is to provide feed and additive which have an improved action that activates biological functions of fish and shellfish. Another object of the present invention is to provide a method for producing the feed and additive which have an improved action that activates biological functions of fish and shellfish.

### DISCLOSURE OF THE INVENTION

As far as mammals including humans are concerned, once an oil component is ingested in the form of food or feed, the oil component is decomposed by certain enzymes contained in bile, pancreatic fluid and the like into fatty acids and glycerin, which are then absorbed by the bowels.

On the contrary, aquatic animals such as fish and shellfish have undeveloped enzymatic functions and, instead, they convert ingested oil into a form of microglobules, which are greedily absorbed by the bowel.

The present inventor has a foresight in recognizing the importance of the digestive system inherent to fish and shellfish, and came up with the present invention.

The present invention provides a feed additive, feed comprising the additive, and a method for producing the additive and feed.

The additive of the present invention comprises a multitude of oil globules each having a diameter of less than 50 μm (preferably less than 10 μm). The oil globules are made of edible oil and retain spherical forms. Each oil globule embeds or encapsulates a biologically active substance that activates biological functions of fish and shellfish.

The oil globules composing the inventive additive are simple substances exclusively consisting of oil. Once the oil globules are ingested by fish and shellfish as part of the feed, the oil globules are conveyed to the bowels of the fish and shellfish via the stomachs thereof, and are taken into the intestinal epithelial cells and then absorbed into the body. During the process of absorbing the oil globules, the biologically active substance contained in the oil globules is absorbed into the body together with the oil globules. Then, the biologically active substance exerts its function.

According to an aspect of the present invention, normal feeding may render the biologically activating effect of the additive to fish and shellfish. Therefore, no particular additional operation (e.g. operation using an adjuvant) is required to augment the disease resistance and immunity of fish and shellfish. Moreover, the additive of the present invention may be applied to young fish without any harm, and is stress-free to fish and shellfish. Furthermore, since the oil globules are actively absorbed by the bowels of fish and shellfish, the absorption efficiency of the biologically activating substance is remarkably high.

In the present invention, any kind of oil is usable as far as the oil is indigestible by fish and shellfish. Some of the examples of the oil are fish oils such as liver oil extracted from squid or cod, sardine oil, and anchovy oil, as well as vegetable oils such as rapeseed oil, corn oil, soybean oil, palm oil, and olive oil. A mixture of two or more kinds of the oils may be used. Alternatively, waste oil of edible oil or its equivalent may be used.

A variety of substances may be used as the biologically activating substance depending on the purpose of use. If the breeder wishes to induce the immunity of fish and shellfish to a specific disease, a substance which may cause the disease is utilized. For instance, if the breeder wishes to induce the immunity to a disease attributable to a certain bacteria or virus, inactivated cells or genetically recombinant protein from the pathogenic bacteria or virus is used. If the breeder wishes to induce the immunity to a disease attributable to an endoparasite parasitic on fish or shellfish, powder of pulverized the endoparasite is used.

If the breeder wishes to culture fish and shellfish having such immunity to diseases, in general, it is preferable to use known general immunopotentiating agents and/or dietary supplements. Examples of such substances are a variety of antibodies, interferons, vitamins, hormone preparations, and enzymes. Agaricus and fucoidan may be included as an example of such substances. Needless to say, these should be construed as mere examples. Such substances may be appropriately selected from plant materials, animal materials, synthetic substances, and the like. The biologically activating substance may be used alone or in combination of two or more substances.

It is preferable to keep the oil globules in an emulsion form. This is because oil globules may be uniformly dispersed and retain their spherical forms in the emulsion. The present invention has a feature that the additive is blended with feed in its use. Since an emulsion exhibits a state substantially equivalent to a state in which oil globules are diluted, use of the additive is advantageous because the additive is uniformly dispersed in the feed.

According to the invention, the method for producing the additive comprises the steps of: pretreatment for preparing powder, aqueous solution or emulsion of a biologically active substance; mixing the powder, aqueous solution or emulsion with edible oil treated with lipophilic emulsifier and stirring the mixture to prepare an intermediate mixture solution in which the biologically active substance is uniformly dispersed in the oil; and separating the intermediate mixture solution into myriads of oil globules.

Whether the biologically active substance is in a form of aqueous solution or emulsion in the pretreatment step, the product prepared by this method is in a form of suspension of oil globules. Thus, it is convenient for storage and delivery.

In the step of dispersing the biologically active substance into oil to prepare an intermediate mixture solution, the mixture is preferably stirred using homo-mixer (of course, other devices may be used). Ultrasonic agitation during the process of preparing oil globules has an advantage of being able to easily preparing oil globules with diameter of several μm.

In the process for preparing the additive, the biologically active substance in the intermediate mixture solution is uniformly dispersed in the oil. In the subsequent step of preparing oil globules, the biologically active substance is maintained in the state of being embedded in the oil globules, in other words, the biologically active substance is supported by oil globules. Thus, the inventive method allows commercial production of the additive capable of activating biological functions of fish and shellfish.

The methods for producing feed having an activating effect on the biological functions of fish and shellfish are classified into methods of adding an additive product to a base material for feed or methods of adding an intermediate product of an additive to a base material for feed. The latter method is included in the present invention, in which intermediate mixture solution is prepared based on the above-mentioned method for producing the additive and the oil contained in the intermediate mixture solution is separated into myriads of microglobules of oil. These microglobules are added to a base material for feed.

Preparation of oil globules by separating the intermediate mixture solution into oil microglobules may be easily achieved by atomization of the intermediate mixture solution with a spraying device. In the case where the base material for feed is a pellet type, atomized intermediate mixture solution may be sprayed on the surface of the base material. In the case where the base material for feed is a paste type, atomized intermediate mixture solution may be kneaded into a base material. If the biologically active substance is in an aqueous solution or suspension at the pretreatment step, oil in the intermediate mixture solution is separated as oil globules in the water. In such case, the intermediate mixture solution itself may be kneaded into a paste-type base material.

The inventive method for producing feed will allow for efficient and low-cost production of feed having an improved action that activates biological functions of fish and shellfish.

### BEST MODE FOR CARRYING OUT THE INVENTION

In the following, a preferred embodiment of the present invention is described. First, an example of the method for producing the additive according to the present invention is described.

### (1) EXAMPLE OF PRODUCTION METHOD

As the first step (pretreatment step), a biologically active substance is rendered to such a state that is easily turned into a water soluble or suspended state. For instance, in the case where the biologically activating substance is water-insoluble solid, the substance is pulverized into a fine powdery state. Whether the mixture of the water and the substance is turned into an aqueous solution or a suspension may be determined depending on the chemical property of the substance. The content of the biologically activating substance is not specifically limited.

In the second step, fish oil containing a lipophilic emulsifier is admixed to the aqueous solution or the suspension to yield an intermediate mixture solution of oil phase and water phase. The mixing ratio of the fish oil relative to the intermediate mixture solution varies depending on the kind and/or the quantity of the biologically activating substance. Generally, the mixing ratio of the fish oil relative to the intermediate mixture solution is preferably in the range from 1 to 10 wt. %. When the biologically active substance is a powdery substance, such as antibody, it may be mixed with fish oil. In such case, the ratio of the fish oil to the intermediate mixture solution is 100%.

The mixing ratio of the lipophilic emulsifier to the fish oil is preferably in the range from 1 to 10 wt. %. Particularly, a preferred mixing ratio of the lipophilic emulsifier to the fish oil is about 5 wt. %.

In the third step, the intermediate mixture solution is stirred using a homo-mixer, or subjected to ultrasonic vibrations with an ultrasonic generator to prepare oil in which the biologically active substance is uniformly dispersed.

In the fourth step (or as an independent step), an aqueous solution is prepared that contains one to a few percentage (in other words, less than 10%) of a hydrophilic (water soluble) emulsifier. This aqueous solution is added to the oil prepared in the third step and stirred by ultrasonic vibrations, thereby converting the oil into oil globules. In other words, by this treatment, the suspended state of the suspension of oil globules is stabilized.

The kinds of the lipophilic and hydrophilic emulsifiers are not specifically limited. Preferably, a food additive approved by a public institution (e.g. Federal and Drug Administration, U.S.A.) is recommended in the aspect of safe use. In Japan, for instance, the product available under the trade name of Sunsoft #818R (sold by Taiyo Kagaku Co., Ltd.) is usable as the lipophilic emulsifier, and the product available under the trade name of Sunsoft Q-812S (sold by Taiyo Kagaku Co., Ltd.) is usable as the hydrophilic emulsifier. Both of the products are fatty acid esters and approved as food additives by the Ministry of Health, Labor and Welfare, Japan.

Adding the hydrophilic emulsifier is advantageous in settling the shape of the oil globules. Thus, an additive in the form of the oil-in-water emulsion containing oil globules embedded with the biologically activating substance is produced. In case of preserving the additive, keeping the additive in a refrigerator for storage is desirable.

Then, the additive is added to a base material for feed for fish and shellfish. In case that the base material for feed is in the form of porous dry pellet, for instance, the additive is sprinkled over the feed, or the base material for feed is dipped into the additive solution, whereby the additive is easily adsorbed to the feed. In case that the feed is in the form of paste, it is preferred to mix the additive with the feed and to knead the mixture. Kneading the mixture does not impair the form of the oil globules, and the oil globules exist in the mixture in the form of particles. Thus, the oil globules embedded with the substance are efficiently absorbed in the bowels of fish and shellfish.

### (2) EXPERIMENT 1

Now, examples of the experiments to confirm the effects of the present invention are described.

### Example 1

This experiment was conducted to confirm the effect that activates the immune system of fish against Edwardsiella disease with which flounders or eels are often infected. The experiment was conducted in a fish farm (fishpond) to present factual evidence. Edwardsiella tarda treated with formalin was used as the biologically activating substance for preparing the additive. Briefly, inactivated bacterial cells were obtained by culturing Edwardsiella tarda, removing impurity by centrifugation, killing the cells with formalin, and then removing the formalin by washing. These inactivated cells were used as the material. By treating the above-mentioned method, an oil globule suspension was produced as the additive. The additive was adsorbed to dry-pellet feed for flounders. Thus, feed containing the biologically activating substance derived from Edwardsiella tarda was prepared.

The flounders (3,000 fishes with average fish weight of 100 g) in a fishpond were fed freely with the feed (0.05 ml/100 g fish weight) for 3 days. Three weeks after the final administration, a blood sample was taken from the tail artery of each flounder. The titer of the anti-Edwardsiella tarda antibody in the blood plasma was counted as an agglutination titer. The result is shown in Table 1.

In Table 1, the fish groups 1 and 2 were fed with the inventive feed. Compared with the fish group 3 which have not been administered with the inventive feed, the fish groups 1 and 2 produced anti-Edwardsiella tarda antibody of a remarkably high concentration in the blood plasma thereof. This proves the effect of the additive of the present invention.

The group 4 in Table 1 is a fish group administered with a mixture of formalin-inactivated Edwardsiella tarda cells, liver oil extracted from squid, and an emulsifier. Increase of the agglutination titer was not observed in fish group 4. This implies that no immunostimulatory effect is expected when the biologically active substance is merely mixed with oil and added to the feed.

The above experiments demonstrates that when fish is fed with feed together with inactivated bacterial cells carried by oil globules, the bacterial cells carried by oil globules are efficiently incorporated into the fish body through the intestinal tract to stimulate and activate the immune system of the fish. The titer of the antibody in the fish ranges from 128 to 1024 (2⁷ to 2¹⁰). The experiments revealed that sufficient protective reaction against intrusion of various pathogenic bacteria can be expected to the fish having such a high antibody titer.

### (3) EXPERIMENT 2

The inventors further conducted experiments to confirm the immunological effect of the product of the present invention. The results are shown in Table 2.

In this experiment, an additive in which inactivated Edwardsiella tarda cells are embedded was prepared and infiltrated into a base material for feed for flounders in the form of porous dry pellet. The feed of the present invention thus prepared was administered to flounders. The various conditions for the experiment were the same as in the Experiment 2.

On day 23 after the final administration of the inventive feed, 15 fishes were picked up and blood samples were collected from five flounders among them to determine the antibody titers in the serum. The remaining 10 flounders were intramuscularly injected with live Edwardsiella tarda cells as the attacking bacterium. The fishes were fed for 14 days to verify the survival rate. As the attacking bacterium, a 10⁵ cfu/ml bacterial suspension was used, and 0.3 ml of the suspension was injected.

The comparative example is a group of flounders fed with conventional feed. In the same manner as described above for the flounders administered with the inventive product, 15 flounders were picked up and blood samples were collected from five among them to determine the antibody titers in the serum. The remaining 10 flounders were injected with live Edwardsiella tarda cells and fed for 14 days to verify the survival rate.

As shown in Table 2, 50% of the individuals in the fish group fed with the inventive feed produced survived such harsh conditions as injection with live bacterial cells, indicating a significantly high survival rate.

TABLE 1

| | Fish given oil globules | | | |
|---|---|---|---|---|
| | embedding antigen | | Controls | |
| | Agglutination titer | | Agglutination titer | |
| Fish | Group-1 | Group-2 | Group-3 | Group-4 |
| Fish 1 | 256 | 32 | 16 | 4 |
| Fish 2 | 1024 | 1024 | 16 | 2 |
| Fish 3 | 64 | 256 | 2 | 8 |
| Fish 4 | 64 | 128 | 64 | 16 |
| Fish 5 | 32 | 256 | 4 | 32 |
| Fish 6 | 256 | 128 | | 2 |
| Fish 7 | 128 | 1024 | | 2 |
| Fish 8 | 128 | 1024 | | 16 |
| Fish 9 | 128 | 256 | | 16 |
| Fish 10 | 256 | 32 | | 64 |

1) 3000 fish a group
2) Group-1 10% feed oil + formalin-killed Edwardsiela tarda (10⁹ cfu/ml)
3) Group-2 1% feed oil + formalin-killed Edwardsiela tarda (10⁷ cfu/ml)
4) Group-3 negative control (normal fish)
5) Group-4 Given with a mixture of bacteria, feed oil and emulsifier
6) Titer was examined in 3 weeks after final given
7) Water temperature: 23-25° C.

TABLE 2

| | Fish given oil | | | |
|---|---|---|---|---|
| | globules embedding | | | |
| | inactivated cells | | Comparative Example | |
| Antibody titer | Fish 1 | 1024 | Fish 1 | 2 |
| | Fish 2 | 1024 | Fish 2 | 4 |
| | Fish 3 | 256 | Fish 3 | 2 |
| | Fish 4 | 32 | Fish 4 | 16 |
| | Fish 5 | 64 | Fish 5 | 16 |

| | | The number | The number |
|---|---|---|---|
| | | of deaths | of deaths |
| Days after | 7 | 0 | 0 |
| injection | 8 | 0 | 0 |
| | 9 | 0 | 1 |
| | 10 | 0 | 4 |
| | 11 | 1 | 1 |
| | 12 | 1 | 0 |
| | 13 | 3 | 1 |
| | 14 | 0 | 3 |

| | | |
|---|---|---|
| Death rate | 50% | 100% |

1. An additive for feed to fish and shellfish consisting essentially of oil globules each having a diameter of less than 10 μm wherein the oil globule is made of edible oil which embeds a substance activating biological functions in the living organism of the fish and shellfish, additionally a lipophilic emulsifier, and optionally, water and a hydrophilic emulsifier.

2. The additive according to claim 1, wherein the substance activating the biological function is at least one selected from the group consisting of inactivated pathogenic virus or bacteria for fish or shellfish, powder of pulverized endoparasite parasitic on fish or shellfish and immunopotentiating agent for fish or shellfish.

3. The additive according to claim 1, comprising water, whereby the oil globules embedding the biologically active substance are in the state of an emulsion or suspension in an aqueous medium.

4. Feed for fish and shellfish, comprising the additive of claim 1.

5. A method for producing an additive for feed for fish and shellfish, comprising the steps of:
preparing a powder, an aqueous solution, or an aqueous suspension of a biologically active substance effective for activating the biological functions of fish and shellfish;
preparing an intermediate mixture solution in which the biologically active substance is dispersed in oil by mixing any one of the powder, aqueous solution or aqueous suspension with edible oil supplemented with a lipophilic emulsifier and stirring the mixture until said substance is uniformly dispersed in the oil; and
forming oil globules by separating the oil contained in the intermediate mixture solution into oil microglobules, each having a diameter of less than 50 μm, said additive consisting essentially of said biologically active substance, edible oil, and, lipophilic emulsifier, and optionally water and a hydrophilic emulsifier.

6. The method according to claim 5, wherein the biologically active substance is in the state of an aqueous solution or suspension, wherein the stirring is performed using a homo-mixer in the step of preparing the intermediate mixture solution, wherein the step of forming oil globules is performed by adding a hydrophilic emulsifier and stirring by ultrasonic vibrations, and wherein the product is in the form an emulsion or suspension of oil globules in an aqueous medium.

7. A method for producing an additive for feed for fish and shellfish, comprising the steps of:
preparing a powder, an aqueous solution, or an aqueous suspension of a biologically active substance effective for activating the biological functions fo fish and shellfish;
preparing an intermediate mixture solution in which the biologically active substance is dispersed in oil by mixing any one of the powder, aqueous solution or aqueous suspension with edible oil supplemented with a lipophilic emulsifier; and
adding by separating the oil contained in the intermediate mixture solution into oil microglobules each having a diameter of less than 50 μm, and incorporating the oil microglobules into a base material for feed for fish or shellfish; said additive consisting essentially of said biologically active substance, edible oil and lipophilic emulsifier and optionally, water and a hydrophilic emulsifier.

8. The method according to claim 7, wherein the intermediate mixture solution is atomized into oil microglobules with a spraying device and the microglobules are sprayed on the surface of the base material in the step of adding, if the base material for feed is in the form of pellets, and wherein the intermediate mixture solution is atomized into oil microglobules with a spraying device and the microglobules are kneaded into the base material in the step of adding, if the base material for feed is in the form of paste.

9. The method according to claim 7, wherein the biologically active substance is in the state of an aqueous solution or suspension and the base material for feed is in the form of paste, and wherein the intermediate mixture solution is kneaded into the base material for feed in the step of adding.
